(19) United States
(12) Patent Application Publication
(10) Pub. No.: US 2012/0041253 A1
(43) Pub. Date: Feb. 16, 2012

(54) Protective Apparatus for Chemotherapy Injection Needle

(76) Inventors: Ching-Yang Wu, Linkou Township (TW); Yun-Hen Liu, Guishan Township (TW); Po-Jen Ko, Taipei City (TW)

(21) Appl. No.: 12/853,432

(22) Filed: Aug. 10, 2010

(51) Int. Cl. A61M 36/08

(52) U.S. Cl. 600/5

(57) ABSTRACT

A protective apparatus for a chemotherapy injection needle, includes a coring needle, a plastic film integrated with a hand-held film, and an injection vessel, wherein the coring needle is a through, hollow and metal needle having a needle head bent downwards to form an angle with a needle body which connects to the injection vessel at a rear end. The plastic film has two trapezoidal and protruding wings extending to two sides, and when the two protruding wings are folded to clamp the needle head, the protruding wings' extending length is sufficient to wrap a tip part of the needle head, and positions where the protruding wings wrap the tip part are corresponding to concaved grooves of the needle head, so that the two protruding wings attach closely with each other. Also, the two protruding wings engage with each other by a locking device to secure the protruding wings.

### FIELD OF THE INVENTION

This invention relates to an injection needle for chemotherapy, and more particularly to a protective apparatus for a chemotherapy injection needle with two wing pieces which are large enough to completely clamp a needle point, and the two wing pieces can be secured to avoid contamination.

### BACKGROUND OF THE INVENTION

Recently, cancer has become the leading cause of death. Many patients have to receive chemotherapy to enhance the results of treatment, or tumor recurrence or distant metastasis has occurred. However, the chemotherapy requires a reliable blood vessel pathway to avoid possible venous thrombosis, and to ensure that the patient does not have to repeatedly receive venopunctures to increase the patient's compliance for the treatment. After an totally implantable intravenous port was invented during the 80's, it not only resolved the problem of drug injection, but also made more convenient for blood sampling. However, a conventional needle is a cutting needle which may puncture the diaphragm surface and further damage the intravenous port. Therefore, the totally implantable intravenous port began its clinical applications after the improvement of the needle (from the cutting needle to a coring needle). In order for easily holding and using the chemotherapy injection needle, a pair of hand-held films are added to a rear portion of the injection needle, so it becomes more convenient for the nursing staff to hold the needle and conduct injection. However, after chemotherapy, the annoying needle puncture problem has not yet been resolved.

Therefore, there remains a need for a new and improved protective apparatus for the chemotherapy injection needle to overcome the abovementioned issues to effectively resolve the needle puncture problem.

### SUMMARY OF THE INVENTION

Conventionally, in order for easily holding and using the chemotherapy injection needle, a pair of hand-held films are added to a rear portion of the injection needle, so it becomes more convenient for the nursing staff to hold the needle and conduct injection. However, after chemotherapy, the annoying needle puncture problem has not yet been resolved.

The present invention is to provide a protective apparatus for a chemotherapy injection needle, including:

a coring needle, a protective film integrated with a hand-held plastic film, and an injection vessel, wherein the coring needle is a through, hollow and metal needle having a needle head bent downwards to form an angle with a needle body which connects with the hand-held plastic film and the injection vessel at a rear end, and

a protective plastic film with two protruding wings extending to two sides, the protective plastic film's inner surface having a concaved portion corresponding to the hand-held plastic film, a bottom portion of the concaved portion having a stationary structure, such that when the hand-held plastic film is inserted into the concaved portion, the stationary structure is used to secure the hand-held plastic film to avoid the hand-held film protruding from the inner surface of the protective plastic film, and makes the protective plastic film smooth and flat, wherein each of the two protruding wings has a front end extending toward the needle head and when the two protruding wings are folded to clamp the needle head, the protruding wings' extending length is sufficient to wrap a tip part of the needle head and positions where the protruding wings wrap the tip part are corresponding to concaved grooves of the needle head, so that the two protruding wings attach more closely with each other, wherein the first protruding wing has a protruding buckle of a locking device for engaging purposes, and the second protruding wing has a buckle groove corresponding to the protruding buckle to secure the two protruding wings to avoid detachment, which can reduce the risk of needles puncture during medical process, and further reduce difficulties of subsequent handling of medical wastes.

Comparing with conventional techniques, the protective apparatus in the present invention can reduce the risk of needle puncture during medical process, and further reduce difficulties of subsequent handling of medical wastes. A use case of the present invention is as following:

1. During injection, the user can fold the hand-held plastic film along with the protective film upwards to hold the needle for injection;

2. During removal process, the user can fold the hand-held plastic film along with the protective film upwards and pull it out;

3. Folding the hand-held plastic film along with the protective film downwards to wrap the tip part of the needle head in the preserved grooves; and

4. Locking the locking device.

Meanwhile, the needle and the tip part is completely wrapped by the protective film and the locking device ensures that the protective film is not loosened. Also, the sharp tip part has already been wrapped inside the protective film, such that the incident of needle puncture to either clinical staff or medical waste cleaning staff can be totally avoided.

The present invention together with the above and other advantages may best be understood from the following detailed description of the embodiments of the invention illustrated in the drawings below.

### BRIEF DESCRIPTION OF THE DRAWINGS

FIG. 1 illustrates a three-dimensional view of one embodiment of the present invention.

FIG. 2 is a schematic view of the present invention where the protective film is folded upwards.

FIG. 3 is a schematic view of the present invention where the protective film is folded downwards.

FIG. 4 depicts a schematic view of the locking device in the present invention.

FIG. 5 illustrates a three-dimensional view of another embodiment of the present invention.

FIG. 6 illustrates a three-dimensional view of a further embodiment of the present invention.

### DETAILED DESCRIPTION OF THE INVENTION

The detailed description set forth below is intended as a description of the presently exemplary device provided in accordance with aspects of the present invention and is not intended to represent the only forms in which the present invention may be prepared or utilized. It is to be understood, rather, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described can be used in the practice or testing of the invention, the exemplary methods, devices and materials are now described.

All publications mentioned are incorporated by reference for the purpose of describing and disclosing, for example, the designs and methodologies that are described in the publications which might be used in connection with the presently described invention. The publications listed or discussed above, below and throughout the text are provided solely for their disclosure prior to the filing date of the present application. Nothing herein is to be construed as an admission that the inventors are not entitled to antedate such disclosure by virtue of prior invention.

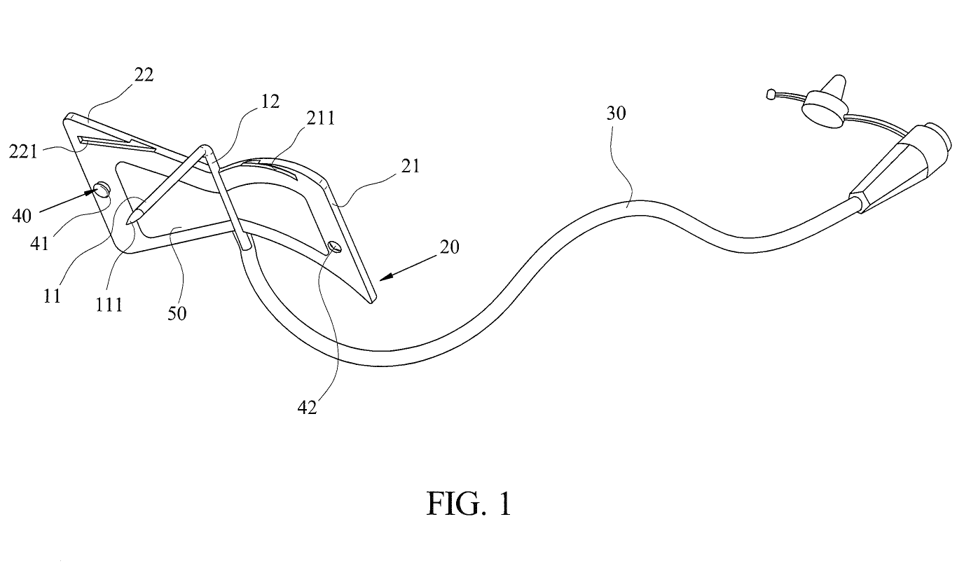

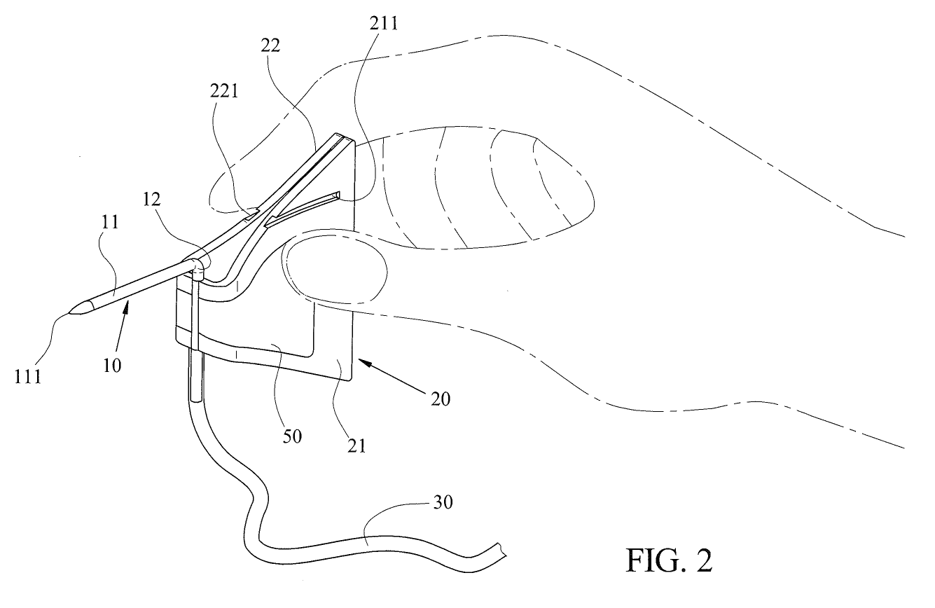

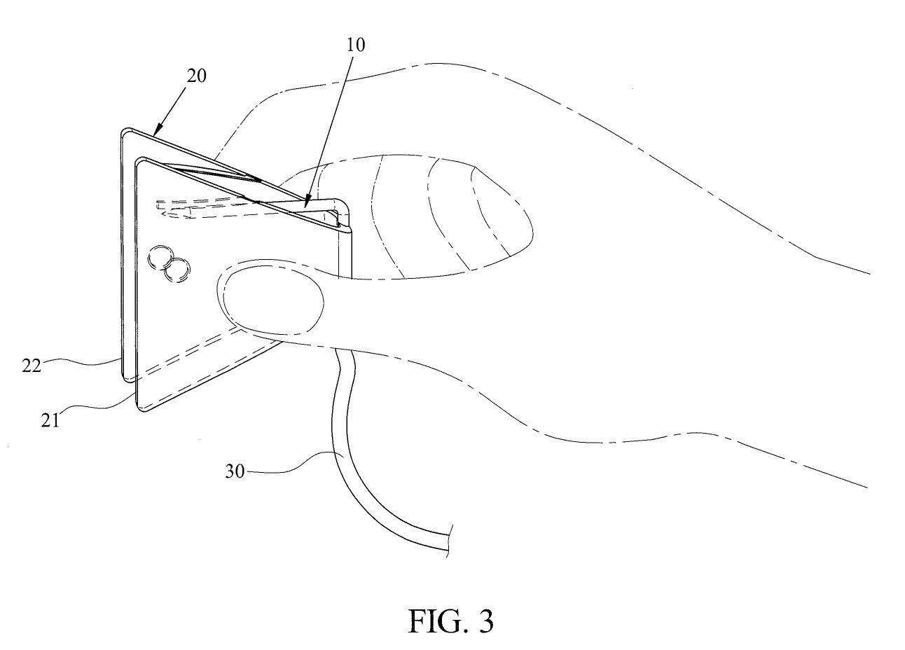

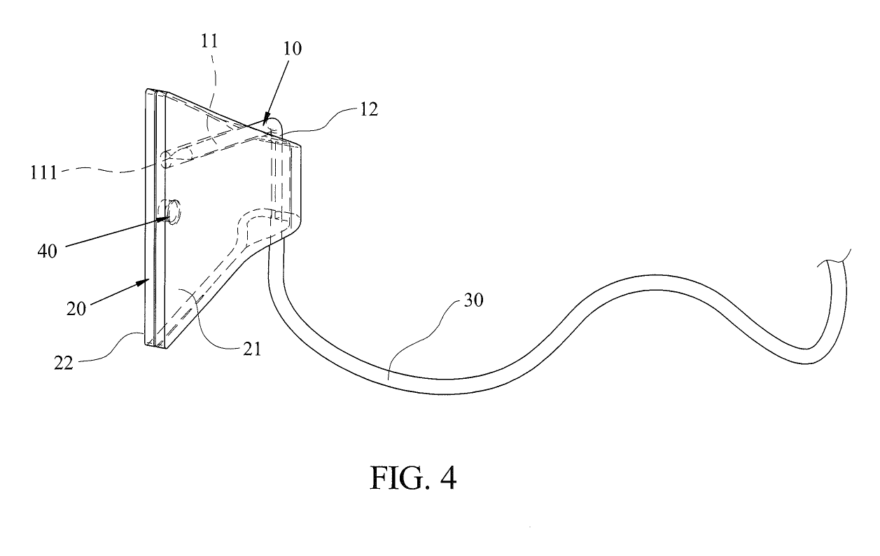

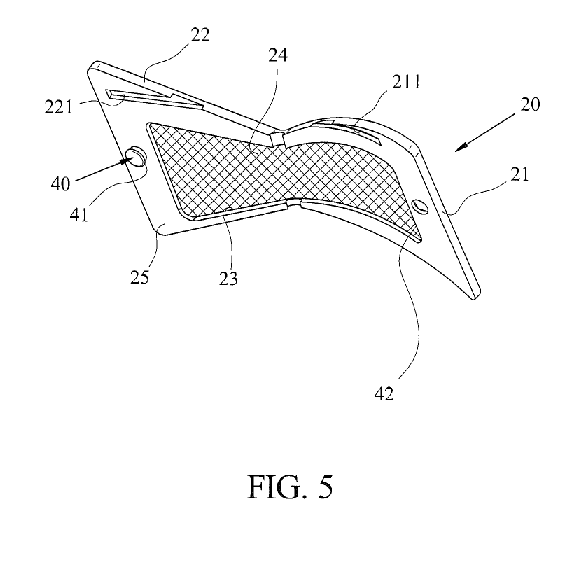

Referring to FIGS. 1 to 5, this invention is adapted to provide a protective apparatus for a chemotherapy injection needle including:

a coring needle 10, a hand-held plastic film 50 and an injection vessel 30, wherein the coring needle 10 is a through, hollow and metal needle having a needle head 11 bent downwards to form an angle with a needle body 12 which connects with the hand-held plastic film 50 and the injection vessel 30 at a rear end, and

a protective plastic film 20 with two protruding wings extending to two sides, the protective plastic film 20′s inner surface 24 having a concaved portion 23 corresponding to the hand-held plastic film 50, a bottom portion of the concaved portion 23 having a stationary structure 24, such that when the hand-held plastic film 50 is inserted into the concaved portion 23, the stationary structure 24 is used to secure the hand-held plastic film 50 to avoid the hand-held film 50 protruding from the inner surface 25 of the protective plastic film 20, and makes the protective plastic film 20 smooth and flat, wherein each of the two protruding wings 21, 22 has a front end extending toward the needle head 11 and when the two protruding wings 21, 22 are folded to clamp the needle head 11, the protruding wings' extending length is sufficient to wrap a tip part 111 of the needle head 11 and positions wrapped by the protruding wings 21, 22 are corresponding to concaved grooves 211, 221 of the needle head 11, so that the two protruding wings 21, 22 attach more closely with each other, wherein the second protruding wing 22 has a protruding buckle 41 of a locking device 40 for engaging purposes, and the first protruding wing 21 has a buckle groove 42 corresponding to the protruding buckle 41. Likewise, the protruding buckle 41 can be formed at the first protruding wing 21 and the buckle groove 42 can be formed at the second protruding wing 22, so that the protruding buckle 41 can engage with the buckle groove 42 to secure the two protruding wings 21, 22 to avoid detachment and reduce the risk of needle puncture during medical processes, and further reduce difficulties of subsequent handling of medical wastes. In one embodiment, the two protruding wings 21, 22 can be the shape of trapezoidal.

The present invention is to provide a protective apparatus for chemotherapy injection needles to reduce the clinical staff's risk of needle puncture and further reduce difficulties of subsequent handling of medical wastes. A method for using the protective device is:

1. During injection, the user can fold the hand-held plastic film 50 along with the protective film 20 upwards to hold the needle for injection;

2. During removal process, the user can fold the hand-held plastic film 50 along with the protective film 20 upwards and pull it out;

3. Folding the hand-held plastic film 50 along with the protective film 20 downwards to wrap the tip part 111 in the preserved grooves 211, 221; and

4. Locking the locking device 40.

In the mean time, the needle head 11 and the tip part 111 are completely wrapped by the protective film 20 and the locking device 40 ensures that the protective film 20 is not loosened. Also, the sharp tip part 111 has already been wrapped inside the protective film 20, such that the incident of needle puncture to either clinical staff or medical waste cleaning staff can be totally avoided. In one embodiment, the stationary structure 24 is glue.

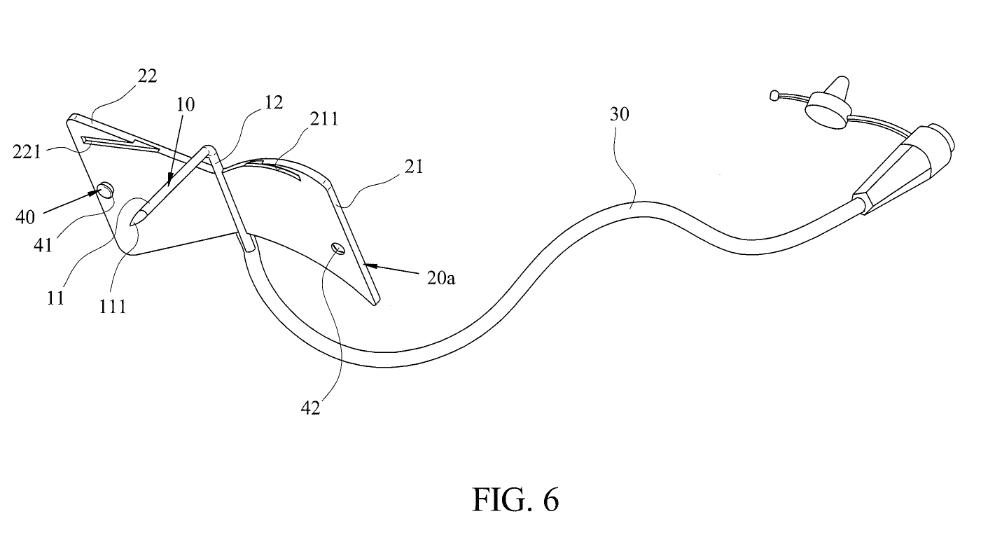

Referring to FIG. 6, a protective device for chemotherapy injection needle includes:

a coring needle 10, a hand-held plastic film and an injection vessel 30, wherein the coring needle 10 is a through, hollow and metal needle having a needle head 11 bent downwards to form an angle with a needle body 12 which connects with the hand-held plastic film and the injection vessel 30 at a rear end, and

a protective plastic film 20a which integrates with the hand-held plastic film to form one unit, and has two protruding wings 21, 22 extending to two sides, wherein each of the two protruding wings 21, 22 has a front end extending toward the needle head 11 and when the two protruding wings 21, 22 are folded to clamp the needle head 11, the protruding wings' extending length is sufficient to wrap a tip part 111 of the needle head 11, and positions where the protruding wings 21, 22 wrap the tip part 111 are corresponding to concaved grooves 211, 221 of the needle head 11, so that the two protruding wings 21, 22 attach more closely with each other, and the two protruding wings 21, 22 engage with each other by a locking device 40 to secure the protruding wings 21, 22 to avoid detachment,

wherein the protruding wings 21, 22 can be the shape of trapezoidal, and

wherein the locking device 40 includes a protruding buckle 41 and a buckle groove 42, and the protruding buckle 41 is engaging with the buckle groove 42 to secure the protruding wings 21, 22 to avoid detachment.

Having described the invention by the description and illustrations above, it should be understood that these are exemplary of the invention and are not to be considered as limiting. Accordingly, the invention is not to be considered as limited by the foregoing description, but includes any equivalents.

What is claimed is:

1. A protective apparatus for a chemotherapy injection needle, comprises:
a protective plastic film with two protruding wings extending to two sides, the protective plastic film's inner surface having a concaved portion, and a bottom portion of the concaved portion having a stationary structure, such that when a hand-held plastic film is inserted into the concaved portion, the stationary structure is used to secure the hand-held plastic film to avoid the hand-held film protruding from the inner surface of the protective plastic film, and makes the protective plastic film smooth and flat, wherein each of the two protruding wings has a front end extending toward the needle head and when the two protruding wings are folded to clamp the needle head, the protruding wings' extending length is sufficient to wrap a tip part of the needle head and positions where the protruding wings wrap the protruding wings are corresponding to concaved grooves of the needle head, so the two protruding wings attach more closely with each other; and the two protruding wings engage with each other by a locking device to secure the two protruding wings to avoid detachment.

2. The protective apparatus for a chemotherapy injection needle of claim 1, wherein a shape of the protruding wings is trapezoidal.

3. The protective apparatus for a chemotherapy injection needle of claim 2, wherein the locking device includes a protruding buckle and a buckle groove, and the protruding buckle and the buckle groove are engaged with each other to secure the two protruding wings.

4. The protective apparatus for a chemotherapy injection needle of claim 1, wherein the stationary structure is glue.

5. A protective apparatus for a chemotherapy injection needle, comprises
a coring needle, a hand-held plastic film and an injection vessel, wherein the coring needle is a through, hollow and metal needle having a needle head bent downwards to form an angle with a needle body which connects with the hand-held plastic film and the injection vessel at a rear end, and
a protective plastic film which integrates with the hand-held plastic film to form one unit, and has two protruding wings extending to two sides, wherein each of the two protruding wings has a front end extending toward the needle head and when the two protruding wings are folded to clamp the needle head, the protruding wings' extending length is sufficient to wrap a tip part of the needle head, and positions where the protruding wings wrap the tip part are corresponding to concaved grooves of the needle head, so that the two protruding wings attach more closely with each other, and the two protruding wings engage with each other by a locking device to secure the protruding wings to avoid detachment.

6. The protective apparatus for a chemotherapy injection needle of claim 5, wherein a shape of the protruding wings is trapezoidal.

7. The protective apparatus for a chemotherapy injection needle of claim 5, wherein the locking device includes a protruding buckle and a buckle groove, and the protruding buckle and the buckle groove are engaged with each other to secure the two protruding wings.
